(19) United States
(12) Patent Application Publication
(10) Pub. No.: US 2010/0030218 A1
(43) Pub. Date: Feb. 4, 2010

(54) Surgical Instrumentation for Forming Threaded Openings in Bone

(75) Inventor: Julien Jean Francis Prevost, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/184,284

(22) Filed: Aug. 1, 2008

(51) Int. Cl. A61B 17/16

(52) U.S. Cl. 606/80

(57) ABSTRACT

Surgical instrument for forming threaded openings in bone including a guide instrument and a tap instrument. The guide instrument includes a guide tube defining a circular passage and a first rotational stop member arranged generally along a first radial axis and defining a first circumferentially-facing stop face. The tap instrument is positioned within the circular passage of the guide tube and includes a distal thread cutting portion and a second rotational stop member arranged generally along a second radial axis and defining a second circumferentially-facing stop face. The second circumferentially-facing stop face of the second rotational stop member is positioned in abutting engagement with the first circumferentially-facing stop face of the first rotational stop member to prohibit further rotation of the tap instrument relative to the guide tube to prevent damage to internal threads formed in bone by continued rotation of the tap instrument.

### FIELD OF THE INVENTION

The present invention relates generally to the field of surgical instruments, and more particularly relates to surgical instrumentation for forming threaded openings in bone.

### BACKGROUND

Various systems have been developed which serve to guide surgical instruments and other devices axially along the interior of a guide tube. Such systems often include drilling and tapping instruments that are used to form threaded openings in bone. When forming threaded openings in bone using a tapping instrument, there is an inherent risk of cutting threads beyond a desired depth and/or stripping or otherwise damaging the newly formed threads via application of excess torque to the tapping instrument.

Thus, there remains a need for improved surgical instrumentation for forming threaded openings in bone. The present invention satisfies this need and provides other benefits and advantages in a novel and unobvious manner.

### SUMMARY

The present invention relates generally to surgical instrumentation for forming threaded openings in bone. While the actual nature of the invention covered herein can only be determined with reference to the claims appended hereto, certain forms of the invention that are characteristic of the preferred embodiments disclosed herein are described briefly as follows.

In one form of the present invention, surgical instrumentation is provided for forming a threaded opening in bone and generally includes a guide instrument and a tap instrument. The guide instrument includes a guide tube defining a circular passage and a first circumferentially-facing stop face positioned at a circumferential location about the circular passage. The tap instrument is positioned within the circular passage of the guide tube and includes a distal thread cutting portion and a second circumferentially-facing stop face. The second circumferentially-facing stop face of the tap instrument is positioned in abutting engagement with the first circumferentially-facing stop face of the guide tube to prohibit further rotation of the tap instrument relative to the guide tube to prevent damage to internal threads formed in bone by the tap instrument by continued rotation of the thread cutting portion.

In another form of the present invention, surgical instrumentation is provided for forming a threaded opening in bone and generally includes a guide instrument and a tap instrument. The guide instrument includes a guide tube defining a circular passage and a first rotational stop member arranged generally along a first radial axis extending from a central longitudinal axis of the guide tube. The tap instrument is positioned within the circular passage of the guide tube and includes a distal thread cutting portion and a second rotational stop member arranged generally along a second radial axis extending from the central longitudinal axis. The second rotational stop member of the tap instrument is positioned in abutting engagement with the first rotational stop member of the guide tube to prohibit further rotation of the tap instrument relative to the guide tube to prevent damage to internal threads formed in bone by the tap instrument by continued rotation of the thread cutting portion.

In a further form of the present invention, surgical instrumentation is provided for forming a threaded opening in bone and generally includes a guide instrument and a tap instrument. The guide instrument includes a guide tube defining a circular passage and a first rotational stop member arranged generally along a first radial axis extending from a central longitudinal axis of the guide tube and defining a first circumferentially-facing stop face positioned at a circumferential location about the circular passage. The tap instrument is positioned within the circular passage of the guide tube and includes a distal thread cutting portion and a second rotational stop member arranged generally along a second radial axis extending from the central longitudinal axis and defining a second circumferentially-facing stop face. The second circumferentially-facing stop face of the second rotational stop member is positioned in abutting engagement with the first circumferentially-facing stop face of the first rotational stop member to prohibit further rotation of the tap instrument relative to the guide tube to prevent damage to internal threads formed in bone by the tap instrument by continued rotation of the thread cutting portion.

It is one object of the present invention to provide improved surgical instrumentation for forming threaded openings in bone. Further objects, features, advantages, benefits, and aspects of the present invention will become apparent from the drawings and description contained herein.

### BRIEF DESCRIPTION OF THE DRAWINGS

FIG. 1 is a perspective view of a system for forming threaded openings in bone according to one form of the present invention.

FIG. 2 is a perspective view of a guide tube portion arranged at one end of the guide instrument illustrated in FIG. 1.

FIG. 3 is a perspective view of a distal portion of the tap instrument illustrated in FIG. 1.

FIG. 4 is a perspective view of the distal portion of the tap instrument illustrated in FIG. 3 rotatably engaged with the guide tube portion illustrated in FIG. 2.

FIG. 5 is a perspective view of a distal portion of a tap instrument according to another embodiment of the present invention.

FIG. 6 is a perspective view of a drill instrument for use in association with the system illustrated in FIG. 1.

### DESCRIPTION OF THE PREFERRED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is hereby intended, and that alterations and further modifications to the illustrated devices and/or further applications of the principles of the invention as illustrated herein are contemplated as would normally occur to one skilled in the art to which the invention relates.

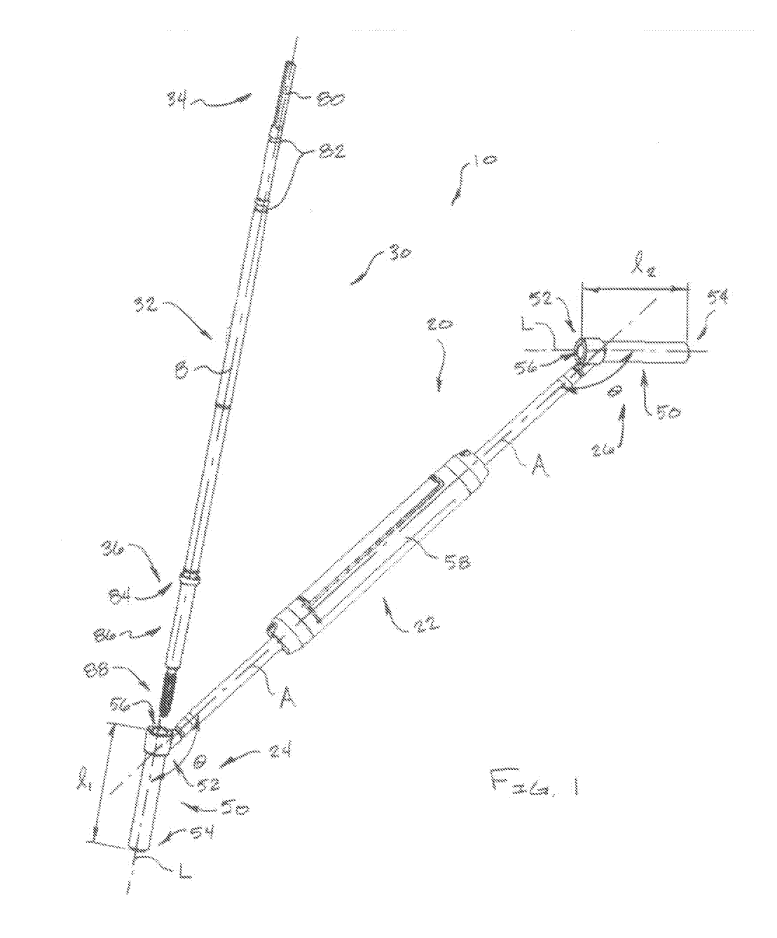

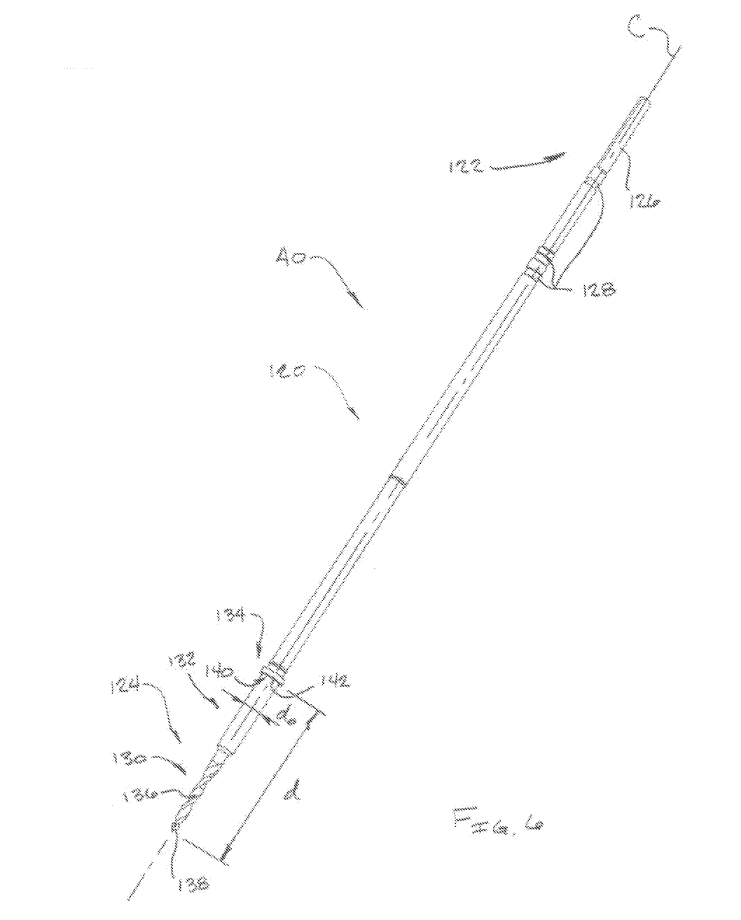

Referring to FIG. 1, shown therein is a system including surgical instrumentation 10 according to one form of the present invention for forming threaded openings in bone. In the illustrated embodiment, the surgical instrumentation 10 includes a guide instrument 20 configured for guiding surgical instruments along an axial passage, and a tap instrument 30 configured for cutting threads along an opening in bone. Additionally, the surgical instrumentation 10 may include a drill instrument 40 (FIG. 6) configured for drilling an opening in bone, an inserter instrument (not shown) configured for inserting an implant to a surgical site, a driver instrument (not shown) configured for driving an implant such as a bone screw into an opening in bone, or other types of surgical instruments that would occur to one of ordinary skill in the art.

The guide instrument 20 generally includes an elongate shaft portion 22 extending along a central longitudinal axis A and guide portions 24, 26 arranged at opposite ends of the elongate shaft portion 22. The tap instrument 30 generally includes an elongate shaft portion 32 extending along a central longitudinal axis B and proximal and distal portions 34, 36 arranged at opposite ends of the elongate shaft portion 32. The guide and tap instruments 20 and 30 may be formed of metallic materials including, for example, surgical grade stainless steel, titanium, or any other suitable material. Further details regarding the guide and tap instruments 20 and 30 will be set forth below.

In the illustrated embodiment of the guide instrument 20, the guide portions 24, 26 are similarly configured, each including a guide tube 50 having proximal and distal portions 52, 54 and defining an axial passage 56 extending therethrough and arranged along a central longitudinal axis L. The guide instrument 20 may also be provided with a handle or gripping portion 58 arranged along a central region of the elongate shaft portion 22 to facilitate grasping and manipulation of the guide instrument 20 by a surgeon or other medical personnel. The handle 58 may be covered by a silicone sleeve or overmolding to facilitate more secure gripping by the surgeon. The guide tubes 50 are operatively attached to opposite ends of the elongate shaft portion 22 with the central longitudinal axis L of the guide tube 50 arranged at an oblique angle θ relative to the longitudinal axis A of the elongate shaft portion 22. In one embodiment, the oblique angle θ is approximately 135 degrees. However, it should be understood that other angles θ are also contemplated. In one embodiment, the guide tubes 50 associated with the guide portions 24, 26 have at least one differing characteristic. In the illustrated embodiment, the guide tubes 50 are provided with different overall lengths l₁ and l₂ to form openings in bone having different depths. In another embodiment, the axial passages 56 defined by the guide tubes 50 may be provided with different inner diameters to receive and guide surgical instruments having different outer diameters. In other embodiments, the guide instrument 20 may be provided with a single guide tube 50 positioned at either end of the elongate shaft portion 22.

Additionally, in the illustrated embodiment of the guide instrument 20, the guide tubes 50 are non-removably and non-movably attached to the ends of the elongate shaft portion 22. However, in other embodiments, the guide tubes 50 may be removably attached to the elongate shaft portion 22 to allow for removal and reattachment of guide tubes having different characteristics (i.e., different tube length, different inner diameters, etc.), and/or the guide tubes 50 may be movably attached to the elongate shaft portion 22 to allow the guide tubes 50 to be positioned at varying angles θ relative to the elongate shaft portion 22 and/or at different rotational positions relative to the elongate shaft portion 22. In still other embodiments, the portions of the elongate shaft 22 extending away from the guide tubes 50 may be removably and/or movably attached to the handle or gripping portion 58.

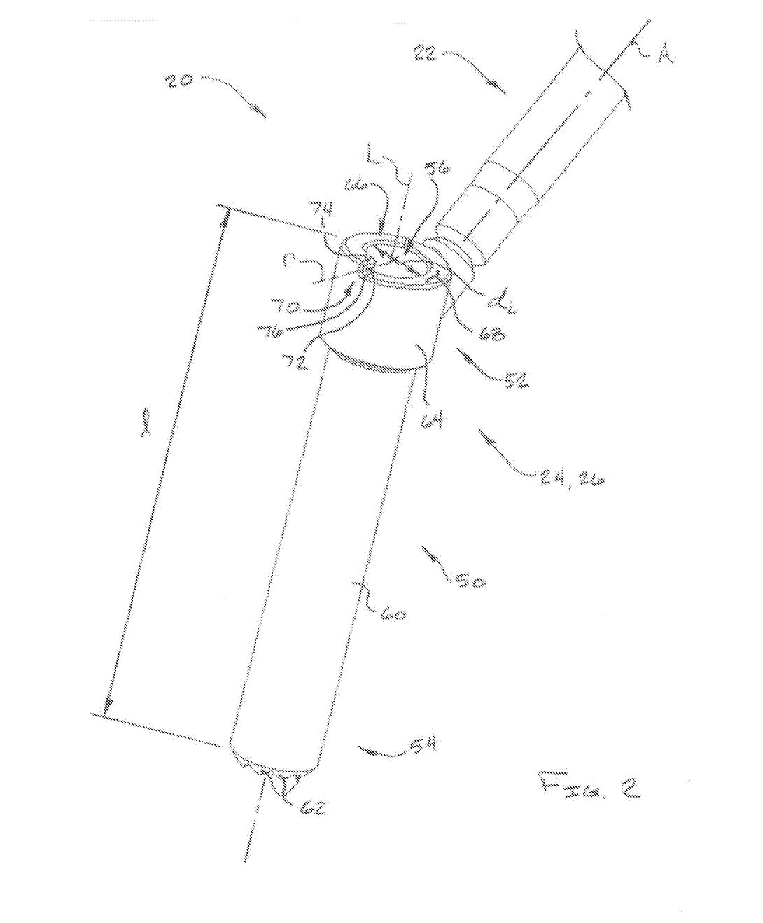

Referring now to FIG. 2, shown therein is an end portion of the guide instrument 20 illustrating further features associated with the guide portions 24, 26, and more specifically the guide tubes 50. As indicated above, the guide tube 50 each have proximal and distal portions 52, 54 and define an axial passage 56 extending therethrough and arranged along a central longitudinal axis L. In the illustrated embodiment, the guide tube 50 has a cylindrical side wall 60 having a length, and the axial passage 56 defined by the side wall 60 has an inner diameter dᵢ. In one embodiment, the inner diameter dᵢ is substantially uniform and constant along substantially the entire length of the side wall 60. The distal end portion 54 of the guide tube 50 may be provided with anchor elements 62 to facilitate more secure engagement with bone. In the illustrated embodiment, the anchor elements 62 are configured as triangular-shaped teeth extending axially from and arranged circumferentially about the distal end of the side wall 60. It should be understood that any number of anchor elements 62 may be provided along the distal end of the side wall 60, and that other configurations of anchor elements are also contemplated including, for example, spikes, surface roughening or knurling, an end surface that tapers to a point and which extends annularly about the distal end of the side wall 60, or any other configuration suitable for anchoring to bone. It should further be understood that in other embodiments, the distal end of the side wall 60 may be blunt or substantially smooth.

In the illustrated embodiment of the guide instrument 20, the proximal end portion 52 of the guide tube 50 is provided with an annular flange or ring 64 defined by an enlarged portion of the side wall 60 and extending circumferentially about the axial passage 56. The annular ring 64 defines a proximal annular end 66 having a proximally-facing end surface 68. The annular ring 64 further defines a radial shoulder or stop 70 having a circumferentially-facing stop face 72, the purpose of which will be discussed below. The radially-extending shoulder 70 is positioned at a circumferential location along the proximal annular end 66, with the circumferentially-facing stop face 72 preferably arranged along a radial axis r extending from the central longitudinal axis L. In a more specific embodiment, the stop face 72 extends along a plane including both the radial axis r and the central longitudinal axis L. In the illustrated embodiment, the stop face 72 is substantially flat and planar. However, it should be understood that other shapes and configurations of the stop face 72 are also contemplated. Moreover, although the illustrated embodiment of the guide tube 50 includes a single radial shoulder or stop 70, it should be understood that in other embodiments, the guide tube 50 may be provided with multiple radial shoulders or stops 70 positioned at various circumferential locations along the annular end 66. Furthermore, although the illustrated embodiment of the guide tube 50 depicts the radial shoulder 70 as being positioned at the proximal annular end 66 of the guide tube 50, in other embodiments, the shoulder 70 may be positioned at other positions and locations along the length of the guide tube 50.

Additionally, in the illustrated embodiment of the guide tube 50, the proximally-facing end surface 68 has an at least partially helical-shaped configuration extending helically about the central longitudinal axis L. In the illustrated embodiment, a first portion of the proximally-facing end surface 68 extending from the proximal end 74 of the stop face 72 and about approximately 90° relative to the longitudinal axis L is substantially planar. In one embodiment, the first planar portion of the proximally-facing end surface 68 extends along a plane that is substantially normal or perpendicular to the longitudinal axis L. Additionally, a second portion of the proximally-facing end surface 68 extending from the first planar portion and about approximately 270° relative to the longitudinal axis L to the distal end 76 of the stop face 72 has a helical configuration. However, it should be understood that other shapes and configurations of the proximally-facing end surface 68 are also contemplated, including embodiments where the helical-shaped portion of the proximally-facing end surface 68 extends less than 270° or greater than 270° about the longitudinal axis L. Furthermore, in the illustrated embodiment, the planar portion and the helical-shaped portion of the end surface 68 are substantially flat. However, in other embodiments, at least a portion of the proximally-facing end surface 68 may be curved or arcuate-shaped.

Additionally, the guide tube 50 defines a length l extending from the distal end to the proximal end of the guide tube 50, the purpose of which will be discussed below. In one embodiment, the annular ring 64 associated with the proximal end portion 52 of the guide tube 50 is non-movably attached and/or formed integral with the guide tube 50 so as to define a fixed and non-variable length l of the guide tube 50 between the proximally-facing end surface 68 and the distal-most end of the guide tube 50. However, in another embodiment, the annular ring 64 may be movably attached to the guide tube 50 so as to define a variable length l between the proximally-facing end surface 68 and the distal-most end of the guide tube 50. Additionally, the annular ring 64 may be locked or fixed at a particular position along the longitudinal axis L via a lock member such as, for example, a set screw, to define a select length l.

Referring once again to FIG. 1, as indicated above, the tap instrument 30 extends generally along a central longitudinal axis B and includes an elongate shaft portion 32 and proximal and distal portions 34, 36 arranged at opposite ends of the elongate shaft portion 32. The proximal portion 34 is configured for engagement with a handle or actuator (not shown). In the illustrated embodiment, the proximal portion 34 has a triangular-shaped configuration including three flattened or truncated regions 80 arranged symmetrically about the longitudinal axis B for rotatable engagement with corresponding portions of a handle or actuator. However, it should be understood that other suitable shapes and configurations of the proximal portion 34 of the tap instrument 30 are also contemplated. Additionally, the proximal portion 34 of the tap instrument 30 may be provided with a number of annular grooves or recessed areas 82 to facilitate engagement with a handle, actuator or other devices.

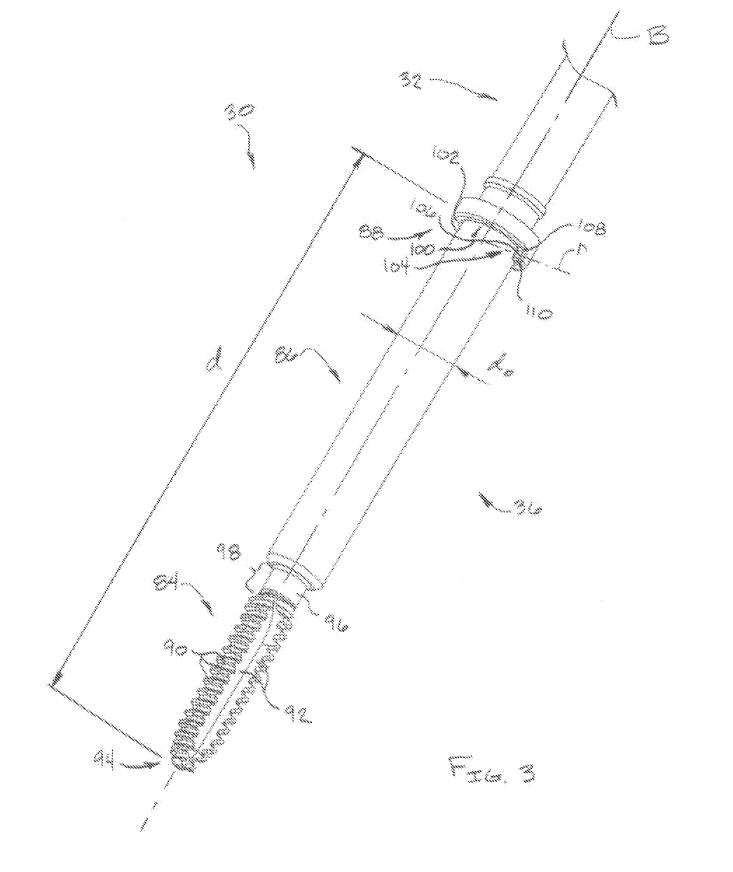

Referring now to FIG. 3, shown therein are features associated with the distal portion 36 of the tap instrument 30. The distal portion 36 of the tap instrument 30 generally includes a cutting region 84, a guiding region 86 and a stop member 88. In the illustrated embodiment, the cutting region 84 includes a helical cutting thread 90 that is circumferentially interrupted by one or more axially-extending cutting flutes 92 which define multiple thread cutting elements. In one embodiment, the cutting region 84 includes three axially-extending cutting flutes 92 extending across the helical thread 86 and arranged generally symmetrical configuration about the longitudinal axis B, with each of the cutting flutes 92 including flute surfaces arranged at approximately a 90° angle relative to one another. Additionally, the cutting region 84 may be provided with a tapered distal end 94 to facilitate insertion into bone. Although a particular configuration of the cutting region 84 has been illustrated and described herein, it should be understood that other configurations of the cutting region 84 are also contemplated. It should also be understood that the distal portion 36 of the tap instrument 30 may be provided with self-drilling features such that the tap instrument 30 may duly serve to form an opening in bone and to cut threads along the formed opening.

In the illustrated embodiment of the tap instrument 30, the guiding region 86 includes a circular outer surface defining an outer diameter dₒ sized slightly smaller but in relatively close tolerance with the inner diameter dᵢ of the axial passage 56 in the guide tube 50. In this manner, the tap instrument 30 may be guided along the central longitudinal axis L of the guide tube 50 as the cutting region 84 and the guiding region 86 are axially displaced along the axial passage 56. The cutting region 84 is connected to the guiding region 86 by a stem portion 96 having an outer diameter sized somewhat smaller than the outer diameter dₒ of the guiding region 86 so as to define an annular groove 98 positioned between the cutting region 84 and the guiding region 86. The annular groove 98 provides a space or pocket between the tap instrument 30 and the inner wall of the guide tube 50 to form a receptacle for receiving bone chips or debris as the cutting region 84 cuts threads into an opening in bone.

In the illustrated embodiment of the tap instrument 30, the stop member 88 is configured as an enlarged annular flange or ring extending circumferentially about the guiding portion 86 and includes a distal annular end 100 having a distally-facing end surface 102. The distal annular end 100 is preferably configured substantially complementary to the proximal annular end 66 defined by the annular ring portion 64 of the guide tube 50 (FIG. 2). In other words, the distal annular end 100 of the stop member 88 is in essence a reverse configuration of the proximal annular end 66 of the guide tube 50. Specifically, the features associated with the distal annular end 100 of the stop member 88 are configured to matingly engage or interlock with the features associated the proximal annular end 66 of the guide tube 50. Additionally, in the illustrated embodiment, the stop member 88 defines a radial shoulder or stop 104 having a circumferentially-facing stop face 106, the purpose of which will be discussed below. The radial shoulder 104 is positioned at a circumferential location along the distal annular end 100 of the stop member 88, with the circumferentially-facing stop face 106 preferably arranged along a radial axis r extending from the central longitudinal axis B. In a more specific embodiment, the stop face 106 extends along a plane including both the radial axis r and the central longitudinal axis B. In one embodiment, the stop face 106 is substantially flat and planar. However, it should be understood that other shapes and configurations of the stop face 106 are also contemplated. Moreover, although the illustrated embodiment of the stop member 88 includes a single radial shoulder or stop 104, it should be understood that in other embodiments, the stop member 88 may be provided with multiple radial shoulders or stops positioned at various circumferential locations along the annular end 100.

Additionally, in the illustrated embodiment of the stop member 88, the distally-facing end surface 102 has an at least partially helical-shaped configuration extending helically about the central longitudinal axis B. In the illustrated embodiment, a first portion of the distally-facing end surface 102 extending from the distal end 110 of the stop face 106 and about approximately 90° relative to the longitudinal axis B is substantially planar. In one embodiment, the first planar portion of the distally-facing end surface 102 extends along a plane that is substantially normal or perpendicular to the longitudinal axis B. Additionally, a second portion of the distally-facing end surface 102 extending from the first planar portion and about approximately 270° relative to the longitudinal axis B to the proximal end 108 of the stop face 106 has a helical configuration. However, it should be understood that other shapes and configurations of the distally-facing end surface 102 are also contemplated, including embodiments where the helical-shaped portion of the distally-facing end surface 102 extends less than 270° or greater than 270° about the longitudinal axis B. Furthermore, in the illustrated embodiment, the planar portion and the helical-shaped portion of the end surface 102 are substantially flat. However, in other embodiments, at least a portion of the distally-facing end surface 102 may be curved or arcuate-shaped.

As shown in FIG. 3, the stop member 88 is positioned at a select axial location along the longitudinal axis B so as to define a distance d between the distally-facing end surface 102 and the distal-most end of the cutting region 84, the purpose of which will be discussed below. In one embodiment, the stop member 88 is non-movably attached to the elongate shaft portion 32 or the guiding portion 86 so as to define a fixed and non-variable distance d IS between the distally-facing end surface 102 and the distal-most end of the cutting region 84. However, in another embodiment, the stop member 88 may be movably attached to the elongate shaft portion 32 or the guiding portion 86 so as to define a variable distance d between the distally-facing end surface 102 and the distal-most end of the cutting region 84. Additionally, the stop member 88 may be locked or fixed at a particular position along the longitudinal axis B via a lock member such as, for example, a set screw, to define a select distance d.

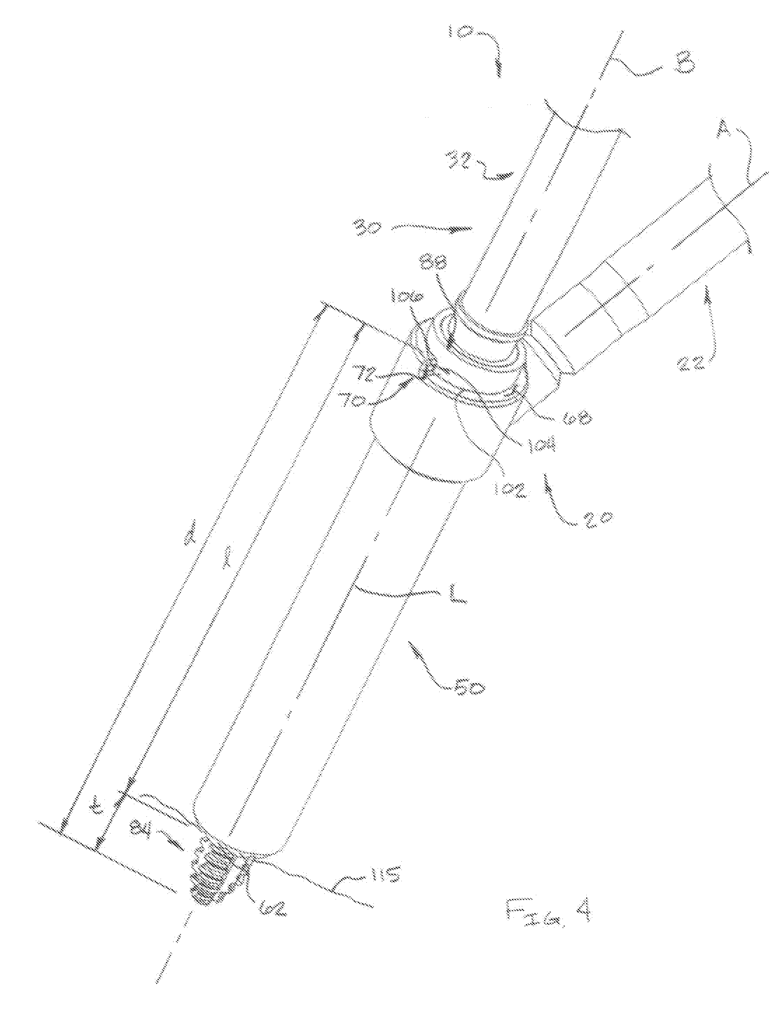

Having described the structural elements and features associated with the guide instrument 20 and the tap instrument 30, reference will now be made to operation of and interaction between the guide instrument 20 and the tap instrument 30 according to one embodiment of the present invention. Referring to FIG. 4, one of the guide tubes 50 of the guide instrument 20 is initially positioned at a surgical site with the central longitudinal axis L of the axial passage 56 aligned with a preformed opening in a bone 115 along which internal threads are to be formed. However, as indicated above, the tap instrument 30 may be provided with self-drilling features so as to duly serve to form the opening in the bone and to cut internal threads along at least a portion of the opening. The anchor elements 62 extending from the distal end of the guide tube 50 are firmly engaged against the outer surface of the bone 115 to inhibit movement of the guide tube 50 relative to the bone 115. The distal portion 36 of the tap instrument 30 is then displaced along the axial passage 56 in a distal direction until the guiding region 86 is guidingly engaged within the axial passage 56 and the distal end 94 of the cutting region 84 is positioned adjacent the bone opening. A rotational force is then applied to the tap instrument 30 to rotate the cutting region 84 about the longitudinal axis B to commence cutting of internal threads along the opening in the bone.

Tapping of the opening in the bone continues until the circumferentially-facing stop face 106 defined by the radial shoulder 104 of the stop member 88 is positioned in abutment against and interlocked with the circumferentially-facing stop face 72 defined by the radial shoulder 70 of the guide tube 50. At this point, continued rotation of the cutting portion 84 of the tap instrument 30 within the opening in the bone is prohibited, thereby preventing the cutting region 84 from stripping or otherwise damaging the internal threads formed along the bone opening. Additionally, since the proximally-facing end surface 68 of the guide tube 50 and the distally-facing end surface 102 of the stop member 88 have a helical-shaped configuration, interference between the stop member 88 and the guide tube 50 is avoided until the circumferentially-facing stop face 106 of the stop member 88 is positioned in abutment against the circumferentially-facing stop face 72 of the guide tube 50.

Furthermore, as should be appreciated, the tapped depth t of the internal threads formed along the opening in the bone 115 by the tap instrument 30 is approximately equal to the difference between the distance d along the distal portion 36 of the tap instrument 30 (measured from the distally-facing end surface 102 and the distal-most end of the cutting region 84) and the length l of the guide tube 50(measured from the proximally-facing end surface 68 and the distal-most end of the guide tube 50). However, as discussed above with regard to the guide instrument 20 and the tap instrument 30, either or both of these instruments may be modified such that the distance d defined by the tap instrument 30 and/or the length l of the guide tube 50 may be variably adjusted, which in turn would allow for variation in the tapped depth t of the internal threads formed along the opening in the bone 115 by the tap instrument 30.

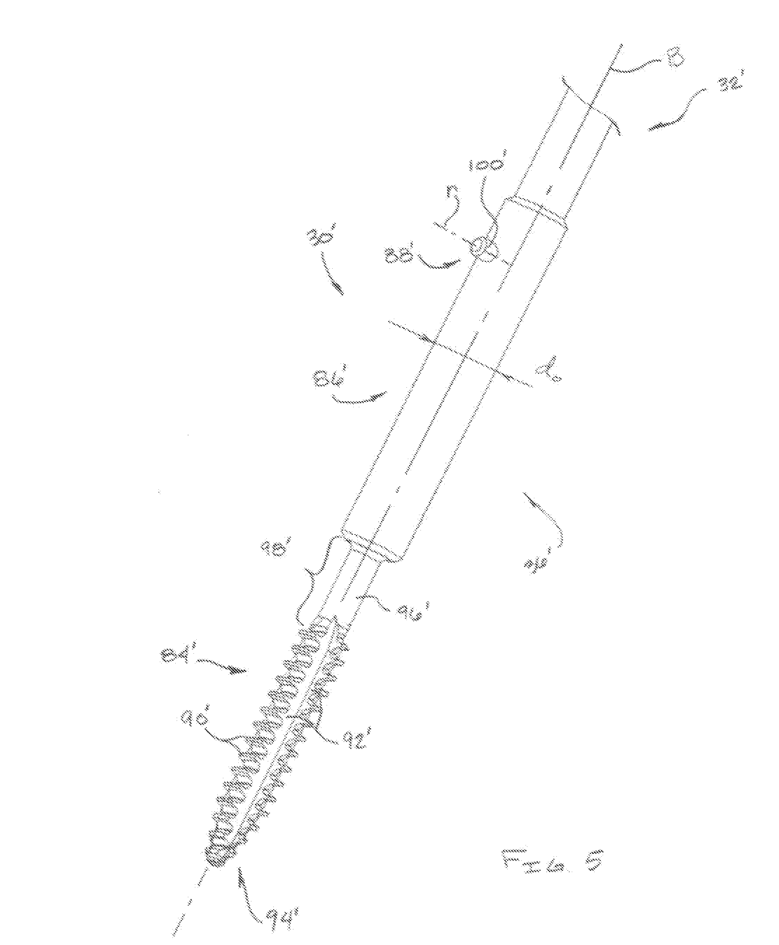

Referring to FIG. 5, shown therein is a tap instrument 30′ according to another embodiment of the present invention. In many respects, the tap instrument 30′ is configured very similar to the tap instrument 30 illustrated and described above. Accordingly, like reference numbers will be used to refer to like features between the tap instruments 30 and 30′. The tap instrument 30′ extends along a central longitudinal axis B and generally includes an elongate shaft portion 32′ and proximal and distal portions 34′, 36′ arranged at opposite ends of the elongate shaft portion 32′. The distal portion 36′ of the tap instrument 30′ generally includes a cutting region 84′, a guiding region 86′ and a stop member 88′. The cutting region 84 includes a helical cutting thread 90′ that is circumferentially interrupted by one or more axially-extending cutting flutes 92′ to define multiple thread cutting elements. Additionally, the cutting region 84′ includes a tapered distal end 94′ to facilitate insertion into bone. In the illustrated embodiment, the guiding region 86′ includes a circular outer surface defining an outer diameter dₒ sized slightly smaller but in relatively close tolerance with the inner diameter dᵢ of the axial passage 56 in the guide tube 50 to axially guide the tap instrument 30′ along the longitudinal axis L. The cutting region 84′ is connected to the guiding region 86′ by a stem portion 96′ having an outer diameter sized somewhat smaller than the outer diameter dₒ of the guiding region 86′ so as to define an annular groove 98′ positioned between the cutting region 84′ and the guiding region 86′ to provide a space or pocket between the tap instrument 30′ and the inner wall of the guide tube 50 for receiving bone chips or debris.

In the illustrated embodiment of the tap instrument 30′, the stop member 88′ is configured as a pin or stem projecting transversely from the guiding region 86′. However, it should be understood that the stop pin member 88′ may extend from other portions of the tap instrument 30′. In one embodiment, the stop pin member 88′ has a circular configuration defining a circular outer surface 100′. As should be appreciated, the stop pin member 88′ provides a radial shoulder or stop that is engaged with the circumferentially-facing stop face 72 associated with the guide tube 50 to prohibit continued rotation of the tap instrument 30′ within the axial passage 56 to prevent the cutting region 84′ from stripping or otherwise damaging the internal threads formed in a bone opening. In the illustrated embodiment, the stop pin member 88′ extends from the outer circular surface of the guiding region 86′ at a circumferential location along the guiding region 86′, with the stop pin member 88′ preferably extending along a radial axis r extending from the central longitudinal axis B. In a more specific embodiment, the stop pin member 88′ extends along a plane including both the radial axis r and the central longitudinal axis B. In the illustrated embodiment, the stop pin member 88′ has a circular configuration. However, it should be understood that other shapes and configurations of the stop pin member 88′ are also contemplated including, for example, elliptical, ovular, rectangular or polygonal shapes and configurations, or any other suitable configuration that would occur to one of ordinary skill in the art.

Moreover, although the illustrated embodiment of the tap instrument 30′ includes a single stop pin member 88′, it should be understood that in other embodiments, the tap instrument 30′ may be provided with multiple stop pin members 88′ positioned at various circumferential locations along the guiding region 86′ or along other portions of the tap instrument 30′. Furthermore, the stop pin member 88′ is positioned at an axial location along the longitudinal axis B so as to define a distance d between the distally-facing outer surface 100′ and the distal-most end of the cutting region 84′. In one embodiment, the stop pin member 88′ is non-movably attached to the guiding portion 86′ of the tap instrument 30′ so as to define a fixed distance d between the distally-facing circular outer surface 100′ and the distal-most end of the cutting region 84′. However, in another embodiment, the stop pin member 88′ may be movably attached to the guiding portion 86′ so as to define a variable distance d between the distally-facing end outer surface 100′ and the distal-most end of the cutting region 84′.

Referring to FIG. 6, shown therein is a drill instrument 40 according to one embodiment of the present invention for use in association with the surgical instrumentation 10. The drill instrument 40 extends along a central longitudinal axis C and generally includes an elongate shaft portion 120 and proximal and distal portions 122, 124 arranged at opposite ends of the elongate shaft portion 120. The proximal portion 122 is configured for engagement with a handle or actuator (not shown). In the illustrated embodiment, the proximal portion 122 is configured similar to the proximal portion 34 of the tap instrument 30, having a triangular-shaped configuration including three flattened or truncated regions 126 for engagement with corresponding portions of a handle or actuator (not shown). Additionally, the proximal portion 122 of the drill instrument 40 may be provided with a number of annular grooves or recessed areas 128 to facilitate engagement with a handle, actuator or other devices. The distal portion 124 of the drill instrument 40 generally includes a drilling region 130, a guiding region 132 and a depth stop member 134. In the illustrated embodiment, the drilling region 130 includes a helical drill flute 136 configured for drilling an opening in bone. Additionally, the drilling region 130 may be provided with a pointed distal end 138 to facilitate penetration into bone. The guiding region 132 includes a circular outer surface defining an outer diameter dₒ sized slightly smaller but in relatively close tolerance with the inner diameter dᵢ of the axial passage 56 in the guide tube 50 so that the drill instrument 40 may be guided along the central longitudinal axis L of the guide tube 50 as the drilling region 130 and the guiding region 132 are axially displaced along the axial passage 56.

In the illustrated embodiment of the drill instrument 40, the depth stop member 134 is configured as an enlarged annular flange or ring and includes a distal annular end 140 having a distally-facing end surface 142. Unlike the stop member 88 associated with the tap instrument 30, the distally-facing end surface 142 of the depth stop member 134 does not have a helical-shaped configuration. Instead, the distally-facing end surface 142 is preferably substantially planar, extending along a plane arranged generally perpendicular or normal to the central longitudinal axis C. Also, unlike the stop member 88, the distally-facing end IS surface 142 of the depth stop member 134 does not include a radially-extending shoulder or a circumferentially-facing stop face. Additionally, the depth stop member 134 is positioned at an axial location along the longitudinal axis C so as to define a distance d between the distally-facing end surface 142 and the distal-most end of the drilling region 130. In one embodiment, the depth stop member 134 is non-movably attached to the guiding portion 132 of the drill instrument 40 so as to define a fixed distance d between the distally-facing end surface 142 and the distal-most end of the drilling region 130. However, in another embodiment, the depth stop member 134 may be movably attached to the guiding portion 132 or to the elongate shaft portion 120 so as to define a variable distance d between the distally-facing end surface 142 and the distal-most end of the drilling region 130. The depth stop member 134 may be locked or fixed at a particular position along the longitudinal axis C via a lock member such as, for example, a set screw, to define a select distance d.

Having described the structural elements and features associated with the drill instrument 40, reference will now be made to operation of and interaction between the guide instrument 20 and the drill instrument 40 according to one embodiment of the present invention. One of the guide tubes 50 of the guide instrument 20 is initially positioned at a surgical site, with the central longitudinal axis L of the axial passage 56 aligned with the portion of bone in which an opening is to be formed. The anchor elements 62 extending from the distal end of the guide tube 50 are firmly engaged with the outer surface of the bone to inhibit movement of the guide tube 50 relative to the bone. The distal portion 124 of the drill instrument 40 is then displaced along the axial passage 56 in a distal direction until the guiding region 132 is guidingly engaged within the axial passage 56 and the distal end of the drilling region 130 is engaged against bone. A rotational force is then applied to the drill instrument 40 to rotate the drilling region 130 about the longitudinal axis C to commence drilling of an opening in the bone. Drilling continues until the distally-facing end surface 142 of the depth stop member 134 engages the planar portion of the proximally-facing end surface 68 of the guide tube 50, thereby preventing further axial displacement of the drill instrument 40 through the guide tube 50. Since the depth stop member 134 does not include a radial shoulder or a circumferentially-facing stop surface engagable with the guide tube 50, continued rotation of the drill instrument 40 is permitted subsequent to engagement of the distally-facing end surface 142 of the depth step member 134 against the planar portion of the proximally-facing end surface 68 of the guide tube 50.

As should be appreciated, the depth of the opening formed in the bone by the drill instrument 40 is approximately equal to the difference between the distance d along the distal portion of the drill instrument 40 (measured from the distally-facing end surface 142 and the distal-most end of the drilling region 130) and the length l of the guide tube 50 (measured from the proximally-facing end surface 68 and the distal-most end of the guide tube 50). As discussed above with regard to the guide instrument 20 and the drill instrument 40, either or both of these instruments may be modified such that the distance d defined by the drill instrument 40 and/or the length l of the guide tube 50 may be variably adjusted, which in turn would allow for variation in the depth of the opening formed in the bone by the drill instrument 40.

As indicated above, other types of surgical instruments may also be used in association with the system 10, including an inserter instrument (not shown) configured for inserting an implant to a surgical site, a driver instrument (not shown) configured for driving an implant such as a bone screw into an opening in bone, or any other type of surgical instrument that would occur to one of skill in the art. It should be understood that these additional surgical instruments may include a stop member configured similar to the stop member 88, 88′ associated with the tap instrument 30, 30′ to prohibit further rotational movement subsequent to engagement of a radial shoulder associated with the stop member 88, 88′ against the radial shoulder 70 defined by the guide tube 50. Alternatively, these additional surgical instruments may include a stop member configured similar to the depth stop member 134 associated with the drill instrument 40 to allow continued rotation of the instrument subsequent to engagement of the distally-facing end surface 142 of the depth step member 134 against the proximal end 66 of the guide tube 50.

While the invention has been illustrated and described in detail in the drawings and foregoing description, the same is to be considered as illustrative and not restrictive in character, it being understood that only the preferred embodiments have been shown and described and that all changes and modifications that come within the spirit of the invention are desired to be protected.

What is claimed is:

1. Surgical instrumentation for forming a threaded opening in bone, comprising:
a guide instrument including a guide tube defining a circular passage extending along a central longitudinal axis, said guide tube further defining a first circumferentially-facing stop face positioned at a circumferential location about said circular passage; and
a tap instrument positioned within said circular passage of said guide tube and extending generally along said central longitudinal axis, said tap instrument including a distal thread cutting portion adapted to cut an internal thread along an opening in bone during rotation of said tap instrument about said central longitudinal axis, said tap instrument defining a second circumferentially-facing stop face; and
wherein said second circumferentially-facing stop face of said tap instrument is positioned in abutting engagement with said first circumferentially-facing stop face of said guide tube to prohibit further rotation of said tap instrument relative to said guide tube to prevent damage to the internal thread by continued rotation of said thread cutting portion.

2. The surgical instrumentation of claim 1, wherein said first circumferentially-facing stop face is arranged generally along a radial axis extending from said central longitudinal axis.

3. The surgical instrumentation of claim 2, wherein said first circumferentially-facing stop face extends generally along a plane including said radial axis and said central longitudinal axis.

4. The surgical instrumentation of claim 1, wherein said first circumferentially-facing stop face is substantially flat and planar.

5. The surgical instrumentation of claim 1, wherein said first circumferentially-facing stop face is defined by a radially-extending shoulder of said guide tube.

6. The surgical instrumentation of claim 1, wherein said first circumferentially-facing stop face extends radially outward from said circular passage.

7. The surgical instrumentation of claim 1, wherein said guide tube includes a proximally-facing surface, at least a portion of said proximally-facing surface having a helical-shaped configuration extending helically about said central longitudinal axis and terminating adjacent said first circumferentially-facing stop face.

8. The surgical instrumentation of claim 7, wherein said proximally-facing surface of said guide tube comprises a proximally-facing annular end surface.

9. The surgical instrumentation of claim 7, wherein said proximally-facing surface of said guide tube extends about said central longitudinal axis from a proximal end of said first circumferentially-facing stop face to a distal end of said first circumferentially-facing stop face.

10. The surgical instrumentation of claim 1, wherein said second circumferentially-facing stop face defined by said tap instrument is arranged generally along a radial axis extending from said central longitudinal axis.

11. The surgical instrumentation of claim 10, wherein said second circumferentially-facing stop face extends generally along a plane including said radial axis and said central longitudinal axis.

12. The surgical instrumentation of claim 11, wherein said second circumferentially-facing stop face extends generally along a plane including said radial axis and said central longitudinal axis.

13. The surgical instrumentation of claim 1, wherein said second circumferentially-facing stop face is substantially flat and planar.

14. The surgical instrumentation of claim 1, wherein said second circumferentially-facing stop face is defined by a radially-extending shoulder of said tap instrument.

15. The surgical instrumentation of claim 1, wherein said tap instrument includes a rotational stop member defining said second circumferentially-facing stop face, said rotational stop member including a distally-facing end surface, at least a portion of said distally-facing end surface extending helically about said central longitudinal axis and terminating adjacent said second circumferentially-facing stop face.

16. The surgical instrumentation of claim 15, wherein said guide tube includes a proximally-facing end surface, at least a portion of said proximally-facing end surface having a helical-shaped configuration extending helically about said central longitudinal axis and terminating adjacent said first circumferentially-facing stop face, said helical-shaped portion of said distally-facing end surface of said rotational stop member matingly engaging said helical-shaped portion of said proximally-facing end surface of said guide tube when said second circumferentially-facing stop face is positioned in abutting engagement with said first circumferentially-facing stop face.

17. The surgical instrumentation of claim 15, wherein said distally-facing end surface of said rotational stop member extends from a proximal end of said second circumferentially-facing stop face to a distal end of said second circumferentially-facing stop face.

18. The surgical instrumentation of claim 1, wherein said tap instrument includes a rotational stop member defining said second circumferentially-facing stop face, said rotational stop member comprising a radially-extending pin member.

19. The surgical instrumentation of claim 18, wherein said pin member has a circular outer cross section, said circular outer cross section having a circular outer surface defining said second circumferentially-facing stop face.

20. The surgical instrumentation of claim 1, wherein said tap instrument includes a circular guiding portion having an outer diameter sized in relatively close tolerance with an inner diameter of said circular passage in said guide tube to guide said tap instrument along said central longitudinal axis as said guiding portion is axially displaced along said circular passage.

21. The surgical instrumentation of claim 1, further comprising a drill instrument including a distal drilling portion and a depth stop member positioned proximal of said drilling portion, said depth stop including a distally-facing end surface positioned in abutting engagement with a proximally-facing end surface of said guide tube to prohibit further axial displacement of said drill instrument in a distal direction relative to said guide tube while permitting continued rotation of said drill instrument within said guide tube.
